(19) United States
(12) Patent Application Publication
(10) Pub. No.: US 2024/0382374 A1
(43) Pub. Date: Nov. 21, 2024

(54) RADIO FREQUENCY OUTPUT APPARATUS CAPABLE OF VIBRATING, AND CONTROL METHOD THEREOF

(71) Applicant: WONTECH Co., Ltd., Daejeon (KR)

(72) Inventors: Jong Won KIM, Seongnam si (KR); Jung Hyun KIM, Seongnam-si (KR); Young Seok SEO, Sejong-si (KR); Seong Cheol CHO, Daejeon (KR)

(21) Appl. No.: 18/466,760

(22) Filed: Sep. 13, 2023

(30) Foreign Application Priority Data
May 18, 2023 (KR) 10-2023-0064051

(51) Int. Cl. A61H 23/02; A61N 1/40

(52) U.S. Cl. CPC A61H 23/02; A61N 1/40; A61H 2201/0153; A61H 2201/0214; A61H 2201/025; A61H 2201/5007; A61H 2201/5043

(57) ABSTRACT

Proposed is a radio frequency output apparatus capable of vibrating and a control method thereof. The apparatus includes: a main body for overall operation control; a handpiece connected to the main body and operated by receiving power from the main body; and a tip attached to a side of the handpiece and in close contact with user's skin, wherein the main body may include: a first power part; a first control part; a first display part; a storage part; an energy generation part; and a cooling part, and the handpiece may include: a second power part; a second control part; a second display part; an energy delivery part; a gas delivery part; and a casing, and the tip may include: a housing; an electrode part; a data collection part; and a chamber, and the tip may further include: a vibration part.

### CROSS REFERENCE TO RELATED APPLICATION

The present application claims priority under 35 U.S.C § 119 to Korean Patent Application No. 10-2023-0064051, filed on May 18, 2023, the entire contents of which is incorporated herein for all purposes by this reference.

### BACKGROUND OF THE INVENTION

### Field of the Invention

The present disclosure relates to a radio frequency output apparatus and a control method thereof and, more particularly, to a radio frequency output apparatus capable of delivering radio frequency energy to the patient's skin while simultaneously transmitting vibrations to the skin.

### Description of the Related Art

Recently, a technology for treating skin conditions by modifying the state of skin tissue or improving tissue characteristics by providing energy to the skin using various energy sources has been widely applied. Skin treatment devices using a variety of energy sources such as laser beams, flash lamps, and ultrasonic waves are being developed, and research on skin treatment devices using RF (radio frequency) energy is also being actively conducted. A specific range of radio frequencies that fall between 3 and 30 megahertz (Mhz) is referred to as high frequency.

When radio frequency energy is provided to the skin surface, molecules constituting skin tissue vibrate and rub against each other whenever the direction of the radio frequency current changes, and deep heat is generated due to rotation, twisting, or collision. The deep heat raises the temperature of skin tissue to reorganize collagen fibers, thereby smoothing wrinkles and improving skin elasticity. In addition, the deep heat has the effect of improving overall condition of the skin, including preventing skin aging by promoting blood circulation in skin tissues.

In order to achieve the above effect, there is a technique of administering radio frequency energy radiation to a deep layer of skin. Yet, when the skin is irradiated with radio frequency energy, pain and hotness due to deep heat may occur, and the degree of discomfort felt varies depending on the patient receiving the treatment. To reduce such discomfort, conventional radio frequency output devices have a vibration unit coupled to the inside of a handpiece housing and transmitting vibrations to the skin, wherein the vibrations generated inside a handpiece due to the operation of the vibrating unit are transmitted into the skin through a tip combined with the handpiece.

However, there is a problem in that efficacy of the vibrations transmitted to a tip end decreases depending on the strength of a user's grip on the handpiece and the degree of adhesion of the tip to the skin.

### SUMMARY OF THE INVENTION

Accordingly, the present disclosure has been made keeping in mind the above problems occurring in the related art, and the present disclosure is intended to provide a radio frequency output apparatus and a control method thereof, in which wires are placed on each side of an electrode plate in contact with the skin to shorten time taken to reach a predetermined temperature, and as a result, duration of radio frequency energy irradiation is reduced but irradiation is repeated several times, and a vibration part is provided on the electrode plate of a tip to enable direct transmission of vibrations into the skin.

In order to achieve the above objective, according to an embodiment of the present disclosure, there is provided a radio frequency output apparatus capable of vibrating, including: a main body for overall operation control; a handpiece connected to the main body and operated by receiving power from the main body; and a tip attached to a side of the handpiece and in close contact with user's skin.

The main body may include: a first power part coupled to the inside of the main body, where power is turned on, and supplying power required for operation of the main body; a first control part coupled to the inside of the main body, operating by receiving power from the first power part, and performing operation control for the main body; a first display part coupled to a side of an upper end of the main body and providing a user interface; a storage part attached to the first control part inside the main body and containing data required for the operation control of the first control part; an energy generation part attached to the inside of the main body, operating by receiving power from the first power part, and generating energy; and a cooling part operating by receiving power from the first power part, and where a gas can is attached so that cooling gas may be delivered to the skin.

The handpiece may include: a second power part coupled to an upper inner surface of the handpiece and enabling an operation of the handpiece by receiving power from the first power part of the main body through wires; a second control part fixed to an inner surface of the handpiece, a part of which protrudes in a shape of a plurality of buttons on an outer surface of the handpiece, operating by receiving power from the second power part, and communicatively connected to the first control part of the main body (direct control of the main body or handpiece is possible by means of the second control part); a second display part coupled to the inner surface of the handpiece, a part of which is exposed to the outer surface of the handpiece, operating by receiving power from the second power part, and providing a user interface related to an operation of the second control part; an energy delivery part that receives the energy generated from the energy generation part of the main body and transfers the received energy to the tip; a gas delivery part that delivers the cooling gas of the gas can attached to the cooling part of the main body toward the skin; and a casing that protects internal components of the handpiece from external shocks.

The tip may include: a housing that protects internal components of the tip from external shocks and includes a coupling device coupled to the handpiece; an electrode part coupled to the inside of the housing, electrically connected with the second power part of the handpiece to receive power from the second power part, electrically connected with the energy delivery part to receive an electrical signal, and irradiates the user's skin with radio frequency energy on the basis of the energy generated from the energy generation part of the main body; a data collection part that measures detailed patient information; and a chamber provided inside the housing, and into which cooling gas for cooling the user's skin is injected.

The tip may further include: a vibration part provided on a side of the electrode part, operating by receiving power generated in the first power part of the main body from the second power part of the handpiece, and transmitting vibrations to the user's skin.

As an example, the electrode part may further include: a third power part to which the power generated in the first power part of the main body is transmitted through the second power part of the handpiece, and connected to a side of the second power part to receive power from the second power part; a first electrode plate and a second electrode plate to which the energy is transmitted from the energy delivery part after the energy generated in the energy generation part of the main body is transmitted through the energy delivery part; and a third electrode plate that receives energy from the first electrode plate and the second electrode plate and irradiates the user's skin with radio frequency energy by coming into contact with the user's skin.

As an example, the main body may further include: a return electrode part that discharges current from a user's body to the outside of the body after irradiating the user's skin with the energy generated in the energy generation part.

In order to achieve the above objective, according to an embodiment of the present disclosure, there is provided a control method of a radio frequency output apparatus capable of vibrating. The method includes: supplying power supplied from a first power part of a main body to a second power part of a handpiece; coupling a gas can to a cooling part of the main body and coupling a tip to the handpiece when power is supplied to the second power part of the handpiece; inputting an energy output signal from a first control part of the main body and a second control part of the handpiece; outputting a microcurrent to an electrode part of the tip after the electrode part of the tip comes into close contact with patient's skin; collecting a patient's impedance value measured after microcurrent is output from a data collection part of the tip; determining, by the first control part, whether the impedance value collected from a patient is 75Ω≤impedance value≤400Ω; starting a vibration output from a vibration part of the tip when the impedance value of the patient falls within a range of 75Ω to 400Ω; starting the vibration output from the vibration part of the tip when the impedance value of the patient falls within the range of 75Ω to 400Ω, and outputting a cooling gas generated in the cooling part of the main body to a chamber of the tip through a gas delivery part of the handpiece; generating radio frequency energy in the energy generation part of the main body and transferring the generated radio frequency energy to the energy delivery part of the handpiece when the cooling gas is output to the chamber of the tip; delivering the radio frequency energy to the patient's skin through the third electrode plate after transferring the radio frequency energy delivered to the energy delivery part of the handpiece to a third electrode plate through a first electrode plate and a second electrode plate of the tip; determining whether the energy output signal is input from the first control part of the main body and the second control part of the handpiece; redoing from the step of outputting the microcurrent to the electrode part of the tip after the electrode part of the tip comes into close contact with patient's skin when it is determined that the energy output signal is input in the step of determining whether the energy output signal is input from the first control part and the second control part; and ending a procedure when it is determined that the energy output signal is not input in the step of determining whether the energy output signal is input from the first control part and the second control part.

As an example, in the step of starting the vibration output from the vibration part of the tip when the impedance value collected from the patient falls within the range of 75Ω to 400Ω, duration of the vibration output may be 2 to 60 seconds.

The method may further include: determining whether the tip is in close contact with the patient's skin when it is determined that the impedance value of the patient does not fall within the range 75Ω to 400Ω in the step of determining whether the impedance value collected from the patient is 75Ω≤impedance value≤400Ω; determining whether a return pad is attached when it is determined that the tip is in close contact with the patient's skin in the step of determining whether the tip is in close contact with the patient's skin; redoing from the step of outputting the microcurrent to the electrode part of the tip after the electrode part of the tip comes into close contact with patient's skin when it is determined that the return pad is attached in the step of determining whether the return pad is attached; redoing from the step of outputting the microcurrent to the electrode part of the tip after the electrode part of the tip comes into close contact with patient's skin when it is determined that the tip is not in close contact with the patient's skin in the step of determining whether the tip is in close contact with the patient's skin; and stopping an operation of a radio frequency output apparatus when it is determined that the return pad is not attached in the step of determining whether the return pad is attached.

As an example, in the step of delivering the radio frequency energy to the patient's skin through the third electrode plate after transferring the radio frequency energy transferred to the energy delivery part of the handpiece to the third electrode plate through the first electrode plate and the second electrode plate of the tip, irradiation of radio frequency energy may be performed 2 to 8 times in succession.

As an example, from the step of outputting the cooling gas from the cooling part of the main body to the chamber of the tip through the gas delivery part of the handpiece to the step of delivering the radio frequency energy to the patient's skin through the third electrode plate after transferring the radio frequency energy transferred to the energy delivery part of the handpiece to the third electrode plate through the first electrode plate and the second electrode plate of the tip may be repeated.

As an example, when repeating the steps of outputting the cooling gas from the cooling part of the main body to the chamber of the tip through the gas delivery part of the handpiece to the step of delivering the radio frequency energy to the patient's skin through the third electrode plate after transferring the radio frequency energy transferred to the energy delivery part of the handpiece to the third electrode plate through the first electrode plate and the second electrode plate of the tip, the cooling gas may be output once, the radio frequency energy may be output at least 4 times continuously, and one output of the cooling gas and 4 outputs of the radio frequency energy may be repeated at least 4 times.

As described above, the present disclosure can reduce the time for an electrode to reach a predetermined temperature, and by shortening duration for one irradiation cycle, burns can be prevented even considering a long irradiation time.

Furthermore, the present disclosure can relieve pain by having a vibration part on an electrode plate of a tip to directly transmit vibrations to the skin.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a block diagram of a radio frequency output apparatus capable of vibrating according to an embodiment of the present disclosure;

FIG. 2 is a schematic diagram of an electrode part of the radio frequency output apparatus capable of vibrating according to the embodiment of the present disclosure;

FIG. 3 is a graph showing radio frequency irradiation time, cooling gas delivery time and vibration output time of the radio frequency output apparatus capable of vibrating according to the embodiment of the present disclosure; and

FIG. 4 is a flowchart of a control method of a radio frequency output apparatus capable of vibrating according to an embodiment of the present disclosure.

### DETAILED DESCRIPTION OF THE INVENTION

Hereinafter, embodiments of the present disclosure will be described in detail with reference to the accompanying drawings so that those skilled in the art can easily carry out the present disclosure. However, the present disclosure may be embodied in many different forms and is not limited to the embodiments described herein. In the drawings, in order to clearly explain the present disclosure, parts irrelevant to the description have been omitted, and similar reference numerals have been assigned to similar parts throughout the specification.

Throughout the specification, when a part is said to be “connected (contacted, coupled)” with another part, this includes not only the case of being “directly connected”, but also the case of being “indirectly connected” with another part in between. In addition, when a part is said to “comprise (include)” a certain component, this means that it may further include other components without excluding other components unless otherwise stated.

The terminology used herein to describe embodiments of the present disclosure is not intended to limit the scope of the present disclosure. The singular expression includes the plural expression unless the context clearly dictates otherwise. It will be further understood that the terms “comprise,” “comprising,” “include,” and/or “including,” when used herein, specify the presence of stated features, numbers, steps, operations, elements, and/or components but do not preclude the presence or addition of one or more other features, numbers, steps, operations, elements, components, and/or groups thereof.

With reference to the accompanying drawings, a radio frequency output apparatus capable of vibrating and a control method thereof according to an embodiment of the present disclosure will be described.

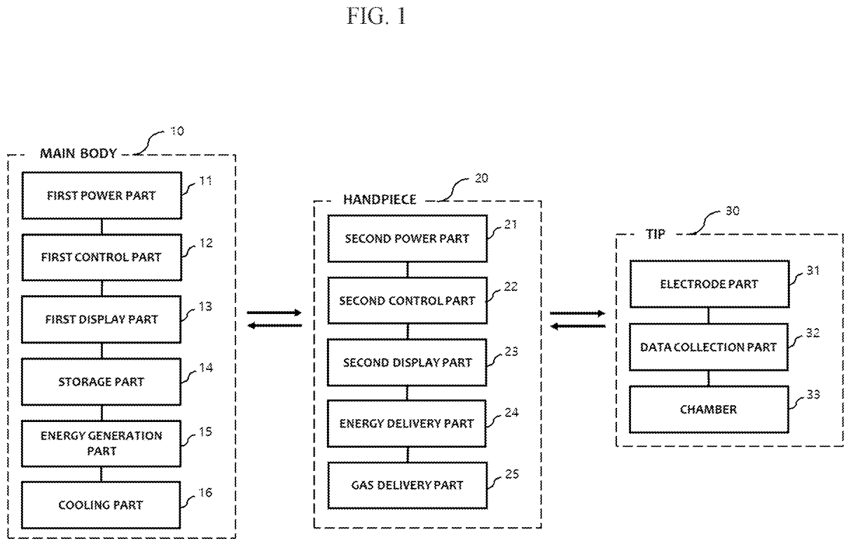

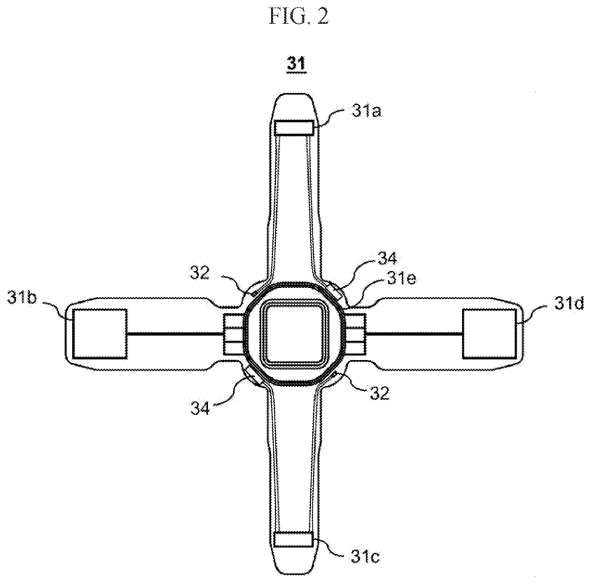

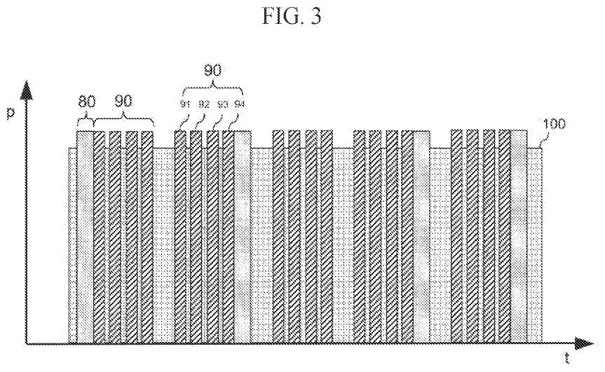

FIG. 1 is a block diagram of a radio frequency output apparatus capable of vibrating according to an embodiment of the present disclosure; FIG. 2 is a schematic diagram of an electrode part of the radio frequency output apparatus capable of vibrating according to the embodiment of the present disclosure; and FIG. 3 is a graph showing radio frequency irradiation time, cooling gas delivery time and vibration output time of the radio frequency output apparatus capable of vibrating according to the embodiment of the present disclosure.

Referring to FIGS. 1 to 3, the radio frequency output apparatus includes: a main body 10 for overall operation control; a handpiece 20 connected to the main body 10 and operated by receiving power from the main body; and a tip 30 attached to one side of the handpiece 20 and in close contact with the user's skin.

The main body 10 includes: a first power part 11 coupled to the inside of the main body 10, where power is turned on, and supplying power required for operation of the main body; a first control part 12 coupled to the inside of the main body 10 and performing operation control by receiving power from the first power part 11; a first display part 13 coupled to one side of the upper end of the main body 10 and provided to an operator and a user for operation control of the first control part 12; a storage part 14 attached to the first control part 12 inside the main body 10 and containing data required for operation control of the first control part 12; an energy generation part 15 attached to the inside of the main body 10 and generating radio frequency energy by receiving power from the first power part 11; and a cooling part 16 capable of attaching a gas can (not shown) to receive power from the first power part 11 and deliver cooling gas to the skin.

The handpiece 20 includes: a second power part 21 coupled to the upper inner surface of the handpiece 20 and enabling the operation of the handpiece 20 by receiving power from the first power part 11 of the main body 10 through wires; a second control part 22 fixed to the inner surface of the handpiece 20, a part of which protrudes in the shape of a plurality of buttons on the outer surface of the handpiece 20, operating by receiving power from the second power part 21, and is interlocked with the first control part 12 of the main body 10 to allow direct control by the user; a second display part 23 coupled to the inner surface of the handpiece 20, a part of which is exposed to the outer surface of the handpiece 20, operating by receiving power from the second power part 21 and providing the user with details of the second control part 22; an energy delivery part 24 that receives energy generated from the energy generation part 15 of the main body 10 and transfers the received energy to the tip 30; a gas delivery part 25 for transferring cooling gas from a gas can of the cooling part 16 of the main body 10; and a casing (not shown) that contains all of the above components and protects the internal components from external shocks.

The tip 30 includes: a housing (not shown) including a configuration of the tip 30, protecting the tip from external shocks, and including a coupling device (not shown) coupled to the handpiece 20; an electrode part 31 coupled to the inside of the housing, transmits power through an electrical connection with the second power part 21 of the handpiece 20, transmits an electrical signal through an electrical connection with the energy delivery part 24, finally irradiates the user's skin with radio frequency energy after the radio frequency energy generated from the energy generation part 15 of the main body 10 is transferred to the energy delivery part 24 of the handpiece 20; a data collection part 32 that operates by receiving power from the electrode part 31 and measures detailed patient information; and a chamber 33 provided inside the housing, and into which gas that cools the user's skin is finally injected through the gas delivery part 25 of the handpiece 20 when the cooling gas is output from the cooling part 16 of the main body 10.

The first display part 23 provides a control UT to the operator, and the first control part 12 is operated by the operator's detailed control.

The storage part 14 includes data on detailed settings required for operation in the first control part 12.

The data stored in the storage part 14 includes data such as radio frequency energy irradiation time, cooling gas delivery time, radio frequency energy output amount, cooling gas output amount, etc., but is not limited thereto.

The cooling part 16 includes: a casing (not shown) to which the cooling gas can (not shown) is attached; a gas output part (not shown) for discharging gas at the end of the casing; and a connection pipe (not shown) connected to one side of the gas output part, and transfers the cooling gas by being connected to the end of the gas delivery part 25 of the handpiece 20 when the gas is emitted from the gas output part.

The second control part 22 of the handpiece 20 is inserted into a hole (not shown) perforated in the casing of the handpiece 20 and protrudes, or is a touch type.

The second display part 23 of the handpiece 20 displays an operating state according to an input value of the first control part 12 of the main body 10 and an input value of the second control part 22 of the handpiece 20 to the user and a patient.

The gas delivery part 25 of the handpiece 20 is connected to one side of the connection pipe of the cooling part 16 of the main body 10, and transfers the cooling gas to the chamber 33 of the tip 30 when gas is discharged from the cooling part 16.

The detailed patient information collected by the data collection part 32 is any one of the patient's impedance value, skin surface temperature, and skin color value, but is not limited thereto.

The electrode part 31 of the tip 30 includes: third power parts 31a and 31c, connected to one side of the second power part 21 to receive power as the power generated in the first power part 11 of the main body 10 is transmitted through the second power part 21 of the handpiece 20; a first electrode plate 31b and a second electrode plate 31d receiving energy from the energy delivery part 24 as the energy generated in the energy generation part 15 of the main body 10 is transmitted through the energy delivery part 24; and a third electrode plate 31e that receives energy from the first electrode plate 31b and the second electrode plate 31d and finally comes into contact with the user's skin to deliver radio frequency energy.

Due to this configuration of the tip 30, energy is simultaneously transferred from the first electrode plate 31b and the second electrode plate 31d to the third electrode plate 31e, so that the time required to reach the predetermined temperature may be shortened.

The main body 10 further includes: a return electrode part (not shown) that irradiates the patient's skin with radio frequency energy generated by the energy generation part 15 and discharges the current transmitted to the patient's body to the outside of the body.

The return electrode part is connected to the main body 10 through wires. To be specific, one end of the return electrode part is attached to the first control part 12 of the main body 10, and the other end of the return electrode part protrudes out of the main body. At the end of one side protruding to the outside of the return electrode plate, an output port having electrodes through which current can flow (not shown) may be further provided.

The output port consists of at least two electrode plates with the same polarity.

In addition, the output port further includes a return pad (not shown) attached to the patient's skin to absorb current absorbed in the patient's body outside the body.

That is, the user's skin is irradiated with the radio frequency energy through the electrode part 31 of the tip 30 as the energy generation part 15 generates radio frequency energy and transmits the generated radio frequency energy to the energy delivery part 24 of the handpiece 20 when power is supplied from the first power part 11 of the main body 10. Thereafter, the irradiated radio frequency energy, that is, radio frequency current is discharged to the first control part 12 through the return pad and the output port electrically connected to the return pad.

A vibration part is further included. The vibration part is provided on one side of the electrode part 31 of the tip 30, operates by receiving power generated in the first power part 11 of the main body from the second power part 21 of the handpiece 20, and transmits vibrations to the patient's skin.

Referring to a graph shown in FIG. 3, the X axis and the y axis respectively show cooling gas delivery 80, radio frequency irradiation 90, and vibration output 100 for irradiation time t and for irradiation amount p.

The cooling gas delivery is characterized in that the delivery time of each pulse is at least longer than the irradiation time of each pulse of the radio frequency irradiation. For example, the delivery time of the cooling gas is 0.2 to 7 seconds and the irradiation time of the radio frequency is 0.1 to 6 seconds, but is not limited thereto.

In addition, the irradiation of radio frequency energy may be performed 2 to 8 times in succession, but is not limited thereto.

The delivery of cooling gas may be performed 1 to 4 times in succession, but is not limited thereto.

Referring to FIG. 3, in the embodiment of the present disclosure, it is preferable to conduct the cooling gas delivery 80 once and then conduct the radio frequency irradiation 90 four times.

The cooling gas delivery 80 is characterized in that the delivery time of each pulse is at least longer than the irradiation time of each pulse of the radio frequency irradiation 90. To be specific, the delivery time of the cooling gas is 0.2 to 7 seconds and the irradiation time of the radio frequency is 0.1 to 6 seconds, but is not limited thereto.

The duration of the vibration output 100 is 2 to 60 seconds, but is not limited thereto.

Next, a radio frequency irradiation method according to an embodiment of the present disclosure will be described in detail with reference to the accompanying drawings.

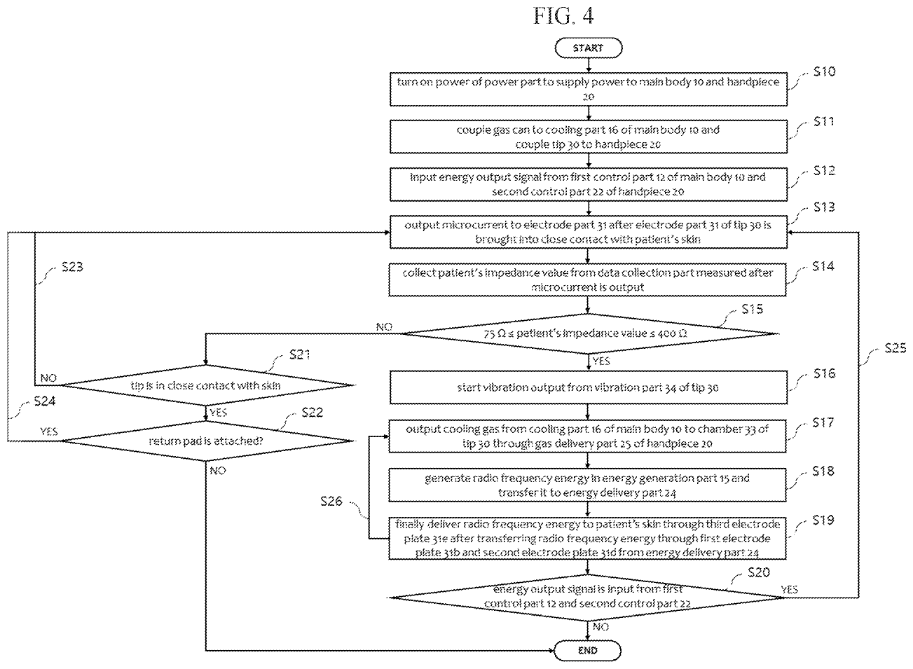

FIG. 4 is a flowchart of a control method of a radio frequency output apparatus capable of vibrating according to an embodiment of the present disclosure.

Referring to FIG. 4, first, when power is supplied from the first power part 11 of the main body 10, the power is supplied (S10) to the second power part 21 of the handpiece 20. When the power is supplied to the second power part 21 of the handpiece 20, the gas can is coupled to the cooling part 16 of the main body 10 and the tip 30 is coupled to the handpiece 20 (S11).

An energy output signal is input (S12) from the first control part 12 of the main body 10 and the second control part 22 of the handpiece 20.

After the electrode part 31 of the tip 30 is brought into close contact with the patient's skin, microcurrent is output (S13) through the electrode part 31 of the tip 30.

The patient's impedance value measured after the microcurrent is output is collected (S14) from the data collection part 32 of the tip 30.

The detailed patient information collected at this time is any one of the patient's impedance value, skin surface temperature, and skin color value, but is not limited thereto.

The first control part 12 of the main body 10 determines (S15) whether the impedance value collected from the patient is 75Ω≤impedance value≤400Ω.

When the patient's impedance value falls within the range of 75Ω to 400Ω, the vibration output starts (S16) from the vibration part of the tip 30.

At this time, the vibration output duration is 2 to 60 seconds, but is not limited thereto.

In the step of determining (S15) whether the impedance value collected from the patient is 75Ω≤impedance value≤400Ω, when the patient's impedance value does not fall within the range of 75Ω to 400Ω, it is determined (S21) whether the tip 30 is in close contact with the patient's skin.

In the step of determining (S21) whether the tip 30 is in close contact with the patient's skin, when it is determined that the tip 30 is in close contact with the skin, it is determined (S22) whether a return pad is attached.

In the step of determining (S22) whether a return pad is attached, when it is determined that the return pad is attached, after closely attaching the electrode part 31 of the tip 30 to the patient's skin, operation starts again (S24) from the step of outputting (S13) the microcurrent to the electrode part 31.

In the step of determining (S21) whether the tip 30 is in close contact with the patient's skin, when it is determined that the tip 30 is not in close contact with the skin, after closely attaching the electrode part 31 of the tip 30 to the patient's skin, operation starts again (S23) from the step of outputting (S13) the microcurrent to the electrode part 31.

In the step of determining (S22) whether a return pad is attached, when it is determined that the return pad is not attached, operation stops. At this time, a notification may be displayed to the user that the return pad is not properly attached.

When the patient's impedance value falls within the range of 75Ω to 400Ω, the vibration output is started (S16) from the vibration part of the tip 30, and the cooling gas generated in the cooling part 16 of the main body 10 is output (S17) to the chamber 33 of the tip 30 through the gas delivery part 25 of the handpiece 20.

At this time, the cooling gas delivery is characterized in that the delivery time is at least longer than the irradiation time of the radio frequency irradiation. The cooling gas delivery time is 0.2 to 7 seconds and the radio frequency irradiation time is 0.1 to 6 seconds, but is not limited thereto.

As described above, when the cooling gas is output to the chamber 33 of the tip 30, the energy generation part 15 of the main body 10 generates radio frequency energy and transfers the generated radio frequency energy to the energy delivery part 24 of the handpiece 20 (S18).

As described above, the radio frequency energy transferred to the energy delivery part 24 of the handpiece 20 is transmitted through the first electrode plate 31b and the second electrode plate 31d of the tip 30, and is finally delivered to the patient's skin through the third electrode plate 31e (S19).

In addition, the irradiation of radio frequency energy may be performed 2 to 8 times in succession, but is not limited thereto.

The steps of outputting the cooling gas from the cooling part 16 of the main body 10 to the chamber 33 of the tip 30 through the gas delivery part 25 of the hand piece 20 (S17) and transmitting radio frequency energy from the energy delivery part 24 through the first electrode plate 31b and the second electrode plate 31d and finally delivering the transmitted radio frequency energy to the patient's skin through the third electrode plate 31e (S19) are repeated (S26) according to the preset number of times. At this time, the preset number of times is characterized in that the cooling gas is output once, the radio frequency energy is continuously irradiated at least 4 times, and the cooling gas output 1 time and the radio frequency energy output 4 times are repeated at least 4 times. In addition, the number of cooling gas outputs and the number of radio frequency energy outputs may vary depending on the set value. In the present embodiment, cooling gas output 1 time and the radio frequency energy output 4 times are repeated at least 5 times.

Next, it is determined (S20) whether an energy output signal is input from the first control part 12 of the main body 10 and the second control part 22 of the handpiece 20.

In the step of determining (S20) whether an energy output signal is input from the first control part 12 and the second control part 22, when it is determined that the energy output signal is input, after the electrode part 31 of the tip 30 comes into close contact with the patient's skin, operation starts again (S25) from the step of outputting (S13) microcurrent to the electrode part 31.

In the step of determining (S20) whether an energy output signal is input from the first control part 12 and the second control part 22, when it is determined that the energy output signal is not input, the procedure ends.

Although the embodiments of the present disclosure have been described in detail above, the scope of the present disclosure is not limited thereto, and various modifications and improvements of those skilled in the art using the basic concept of the present disclosure defined in the following claims also fall within the scope of the present disclosure.

What is claimed is:

1. A radio frequency output apparatus capable of vibrating, the apparatus comprising:
a main body for overall operation control;
a handpiece connected to the main body and operated by receiving power from the main body; and
a tip attached to a side of the handpiece and in close contact with a user's skin, wherein the main body comprises:
a first power part coupled to the inside of the main body, where power is turned on, and supplying power required for operation of the main body;
a first control part coupled to the inside of the main body, operating by receiving power from the first power part, and performing operation control for the main body;
a first display part coupled to a side of an upper end of the main body and providing a user interface;
a storage part attached to the first control part inside the main body and containing data required for the operation control of the first control part;
an energy generation part attached to the inside of the main body, operating by receiving power from the first power part, and generating energy; and
a cooling part operating by receiving power from the first power part, and where a gas can is attached so that cooling gas may be delivered to the skin, and the handpiece comprises:
a second power part coupled to an upper inner surface of the handpiece and enabling an operation of the handpiece by receiving power from the first power part of the main body through wires;
a second control part fixed to an inner surface of the handpiece, a part of which protrudes in a shape of a plurality of buttons on an outer surface of the handpiece, operating by receiving power from the second power part, and communicatively connected to the first control part of the main body, wherein the main body or the handpiece is directly controllable by the second control part;
a second display part coupled to the inner surface of the handpiece, a part of which is exposed to the outer surface of the handpiece, operating by receiving power from the second power part, and providing a user interface related to an operation of the second control part;
an energy delivery part that receives the energy generated from the energy generation part of the main body and transfers the received energy to the tip;
a gas delivery part that delivers the cooling gas of the gas can attached to the cooling part of the main body toward the skin; and
a casing that protects internal components of the handpiece from external shocks, and the tip comprises:
a housing that protects internal components of the tip from external shocks and includes a coupling device coupled to the handpiece;
an electrode part coupled to the inside of the housing, electrically connected with the second power part of the handpiece to receive power from the second power part, electrically connected with the energy delivery part to receive an electrical signal, and configured to irradiate the user's skin with radio frequency energy on the basis of the energy generated from the energy generation part of the main body;
a data collection part that measures detailed patient information; and
a chamber provided inside the housing, and into which cooling gas for cooling the user's skin is injected, and
the tip further comprises:
a vibration part provided on a side of the electrode part, operating by receiving power generated in the first power part of the main body from the second power part of the handpiece, and transmitting vibrations to the user's skin.

2. The apparatus of claim 1, wherein the electrode part further comprises:
a third power part to which the power generated in the first power part of the main body is transmitted through the second power part of the handpiece, and connected to a side of the second power part to receive power from the second power part;
a first electrode plate and a second electrode plate to which the energy is transmitted from the energy delivery part after the energy generated in the energy generation part of the main body is transmitted through the energy delivery part; and
a third electrode plate that receives energy from the first electrode plate and the second electrode plate and irradiates the user's skin with radio frequency energy by coming into contact with the user's skin.

3. The apparatus of claim 1, wherein the main body further comprises:
a return electrode part that allows current remaining in a user's body to be discharged outside of the user's body after the energy generated in the energy generation part is irradiated to the user's skin with.

4. A control method of a radio frequency output apparatus capable of vibrating, the method comprising:
supplying power supplied from a first power part of a main body to a second power part of a handpiece;
coupling a gas can to a cooling part of the main body and coupling a tip to the handpiece when power is supplied to the second power part of the handpiece;
inputting an energy output signal from a first control part of the main body and a second control part of the handpiece;
outputting a microcurrent to an electrode part of the tip after the electrode part of the tip comes into close contact with patient's skin;
collecting a patient's impedance value measured after microcurrent is output from a data collection part of the tip;
determining, by the first control part, whether the impedance value collected from a patient is 75Ω≤impedance value≤400Ω;
starting a vibration output from a vibration part of the tip when the impedance value of the patient falls within a range of 75Ω to 400Ω;
starting the vibration output from the vibration part of the tip when the impedance value of the patient falls within the range of 75Ω to 400Ω, and outputting a cooling gas generated in the cooling part of the main body to a chamber of the tip through a gas delivery part of the handpiece;
generating radio frequency energy in the energy generation part of the main body and transferring the generated radio frequency energy to the energy delivery part of the handpiece when the cooling gas is output to the chamber of the tip;
delivering the radio frequency energy to the patient's skin through the third electrode plate after transferring the radio frequency energy delivered to the energy delivery part of the handpiece to a third electrode plate through a first electrode plate and a second electrode plate of the tip;
determining whether the energy output signal is input from the first control part of the main body and the second control part of the handpiece;
redoing from the step of outputting the microcurrent to the electrode part of the tip after the electrode part of the tip comes into close contact with patient's skin when it is determined that the energy output signal is input in the step of determining whether the energy output signal is input from the first control part and the second control part; and
ending a procedure when it is determined that the energy output signal is not input in the step of determining whether the energy output signal is input from the first control part and the second control part.

5. The method of claim 4, wherein in the step of starting the vibration output from the vibration part of the tip when the impedance value collected from the patient falls within the range of 75Ω to 400Ω, duration of the vibration output is 2 to 60 seconds.

6. The method of claim 4, further comprising:
determining whether the tip is in close contact with the patient's skin when it is determined that the impedance value of the patient does not fall within the range 75Ω to 400Ω in the step of determining whether the impedance value collected from the patient is 75Ω≤impedance value≤400 Ω;
determining whether a return pad is attached when it is determined that the tip is in close contact with the patient's skin in the step of determining whether the tip is in close contact with the patient's skin;
redoing from the step of outputting the microcurrent to the electrode part of the tip after the electrode part of the tip comes into close contact with patient's skin when it is determined that the return pad is attached in the step of determining whether the return pad is attached;
redoing from the step of outputting the microcurrent to the electrode part of the tip after the electrode part of the tip comes into close contact with patient's skin when it is determined that the tip is not in close contact with the patient's skin in the step of determining whether the tip is in close contact with the patient's skin; and
stopping an operation of a radio frequency output apparatus when it is determined that the return pad is not attached in the step of determining whether the return pad is attached.

7. The method of claim 4, wherein in the step of delivering the radio frequency energy to the patient's skin through the third electrode plate after transferring the radio frequency energy transferred to the energy delivery part of the handpiece to the third electrode plate through the first electrode plate and the second electrode plate of the tip, irradiation of radio frequency energy is performed 2 to 8 times in succession.

8. The method of claim 4, wherein from the step of outputting the cooling gas from the cooling part of the main body to the chamber of the tip through the gas delivery part of the handpiece to the step of delivering the radio frequency energy to the patient's skin through the third electrode plate after transferring the radio frequency energy transferred to the energy delivery part of the handpiece to the third electrode plate through the first electrode plate and the second electrode plate of the tip are repeated.

9. The method of claim 8, wherein when repeating the steps of outputting the cooling gas from the cooling part of the main body to the chamber of the tip through the gas delivery part of the handpiece to the step of delivering the radio frequency energy to the patient's skin through the third electrode plate after transferring the radio frequency energy transferred to the energy delivery part of the handpiece to the third electrode plate through the first electrode plate and the second electrode plate of the tip, the cooling gas is output once, the radio frequency energy is output at least 4 times continuously, and one output of the cooling gas and 4 outputs of the radio frequency energy are repeated at least 4 times.
